(19) United States
(12) Patent Application Publication
(10) Pub. No.: US 2010/0112583 A1
(43) Pub. Date: May 6, 2010

(54) BLOOD DIAGNOSIS METHOD FOR DIALYSIS PATIENT AND DIALYSIS MACHINE

(75) Inventors: Eiichiro ICHIISHI, Sendai-shi (JP); Taisuke Baba, Sendai-shi (JP); Yumiko Ishizaki, Sendai-shi (JP); Makoto Ishizaki, Sendai-shi (JP); Kazuhisa Fukushima, Tokyo (JP); Tsuneji Sawai, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: Yokogawa Electric Corporation, Tokyo (JP); Tohoku University, Sendai-shi (JP)

(21) Appl. No.: 12/606,714

(22) Filed: Oct. 27, 2009

(30) Foreign Application Priority Data
Oct. 31, 2008 (JP) 2008-281987

(51) Int. Cl. C12Q 1/68; C12Q 1/02; G01N 1/10

(52) U.S. Cl. 435/6; 435/29; 73/864.31

(57) ABSTRACT

Provided is a blood diagnosis method and a dialysis machine, using a diagnostic marker which is versatile and which can contribute to the improvements in dialysis treatment and the evaluation of clinical effects,
the method including a step for collecting a blood sample from a dialysis patient before and after dialysis; and a step for making a diagnosis regarding the collected blood sample based on a biomarker, wherein the biomarker is identified in advance based on the correlation between the urea clear space (CS) or the cellular membrane clearance (Kc) and profiles of mRNA or proteins.

### BACKGROUND OF THE INVENTION

1. Field of the Invention

The present invention relates to a blood diagnosis method in which a diagnosis is made using blood collected from a dialysis patient, and a dialysis machine suitable for the blood diagnosis method.

2. Description of the Related Art

In dialysis using a dialysis machine (for example, refer to Patent Document 1), it is necessary to select a dialysis membrane suitable for a patient condition, to determine the patient's primary disease, and to follow the clinical progress by observing the prognosis after dialysis and monitoring the risks for complications such as infectious diseases. However, since dialysis patients exhibit remarkably poor functions in terms of excretion and removal of waste products due to a dialysis process inevitably introducing an artificial bias, it is difficult to accurately understand their clinical progress by an evaluation using the same criteria as those applied for healthy individuals. Accordingly, in order to understand a dialysis patient's clinical progress, for example, the following clinical parameters are used as diagnostic markers, in addition to the monitoring of urine volume, body weight, estimated muscle mass, biochemical tests of blood, and removal time for waste products.

### (1) Removal Rate per Hour (HrURR)

The HrURR refers to a removal rate of urea nitrogen per hour, and it is desirable that the HrURR be 30% or less. When the HrURR is equal to or greater than 27.5%, a modification of dialysis treatment is necessary (which is based on the presentation given in the 7th Annual Meeting of Japanese Society for Hemodiafiltration (HDF)).

### (2) Creatinine Generation Rate (% CrG)

A target value is 100% or more for a dialysis patient and is 90% or more for a diabetic dialysis patient. It is necessary to build up muscles and to enhance the creatinine generation rate by appropriately in-taking proteins or exercising.

### (3) Standardized Dialysis Dose (Kt/V)

It is considered that the efficient removal of uremic toxins leads to even more excellent clinical results. It is a well known fact that the prevalence rate increases due to the accumulation of uremic toxins in the body. However, Kt/Vurea which has been used conventionally as a dialysis index does not accurately represent the dialysis dose, that is, the amount of urea removed.

However, a complex numerical calculation based on plural clinical data is required in order to obtain these diagnostic markers. The operation for acquiring the clinical data or the operation for deriving the diagnostic markers on the basis of the clinical data is complicated and thus causes a problem in view of simplicity. Moreover, even among the dialysis patients, their clinical progress varies greatly due to the differences in their primary diseases as well as the individual differences. Accordingly, it is difficult to select dialysis membranes suitable for the dialysis patients or to set dialysis conditions suitable for the dialysis patients. Currently, the details of selection of the diagnostic markers are regarded as know-how and the selection is left up to each medical institution to decide. Therefore, there is a need for a diagnostic marker which is versatile and easy to use in clinical practice. In particular, it is necessary to replace the dialysis membranes at an appropriate time and it is thus difficult to select the replacement time of the membranes as well as to select the type of membranes. Accordingly, if diagnostic information is acquired which can be used as a guideline for making the selection, it will greatly contribute to effective dialysis treatment.

In addition, it has been proved that a factor indicating a patient's nutritive condition such as PEM (Protein Energy Malnutrition) is very important in controlling the clinical effect of a hemodialysis treatment. However, it has been reported that the factor such as PEM has a negative correlation with the conventional diagnostic markers such as the indicator for standardized dialysis dose (i.e., Kt/Vurea). Accordingly, in addition to the conventional diagnostic markers such as Kt/Vurea, there is a need for development of a new index indicating a nutritive condition.

Moreover, it has been reported that various inflammatory cytokines are associated with deterioration in pathological conditions of uremia of patients with end-stage renal failure. Accordingly, if diagnostic markers are found which have a correlation with the generation of inflammatory cytokines, the dialysis treatment may be optimized or the clinical effect may be evaluated appropriately.

[Patent Document 1] Japanese Unexamined Patent Application, First Publication No. Hei 9-10301

[Patent Document 2] Japanese Unexamined Patent Application, First Publication No. 2008-32395

Meanwhile, in addition to the abovementioned Kt/Vurea, the urea clear space (CS) or the cellular membrane clearance (Kc) can be used as an indicator for standard dialysis dose. It has been accepted in Japanese Society for Hemodiafiltration and/or known from experience that the urea clear space (CS) reflects the five-year survival rate and quality of life (QOL) of dialysis patients and the cellular membrane clearance (Kc) reflects nutritional status that strongly affects the clinical results of dialysis patients and their liability. Accordingly, it is thought that they are most suitable to be used as a new indicator for dialysis dose. However, specific equipment as well as the knowledge based on experience is required for measuring the urea clear space (CS) and cellular membrane clearance (Kc). Accordingly, it is not realistic to expect such a procedure to become widespread, in which the conditions of patients are monitored by directly measuring these indicators. For example, for measuring the urea clear space (CS), the entire drain as a result of dialysis over 4 hours is first stored in a tank, and then the amount of toxic substance in the drain needs to be measured. Accordingly, large-scaled equipment as well as complicated procedures is required for the measurements. In addition, although the amount of removed toxin becomes clear from the measurements, variations in the manner in which toxins are removed over time cannot be elucidated.

### SUMMARY OF THE INVENTION

An object of the present invention is to provide a blood diagnosis method and a dialysis machine using a diagnostic marker which is versatile and which can contribute to the improvements in dialysis treatment and the evaluation of clinical effects.

A first aspect of the blood diagnosis method according to the present invention is a blood diagnosis method in which a diagnosis is made using blood collected from a dialysis patient, the method characterized by having: a step for identifying a biomarker in advance based on the correlation between the urea clear space (CS) or the cellular membrane clearance (Kc) and the profiles of mRNA or proteins; a step for collecting a blood sample from a dialysis patient before and after dialysis; and a step for making a diagnosis regarding the collected blood samples based on the identified biomarker.

According to the blood diagnosis method, because a biomarker is identified in advance based on the correlation between the urea clear space (CS) or the cellular membrane clearance (Kc) and the profiles of mRNA or proteins, it is possible to provide a diagnostic marker which is versatile and which can contribute to the improvements in dialysis treatment and the evaluation of clinical effects.

A second aspect of the blood diagnosis method according to the present invention is a blood diagnosis method in which a diagnosis is made using blood collected from a dialysis patient, the method comprising: a step for collecting a blood sample from a dialysis patient before and after dialysis; and a step for making a diagnosis regarding the collected blood sample based on a biomarker, wherein any one of the genes shown below or the combinations thereof is used as the biomarker which is identified from the profiles of mRNA or proteins: TNFSF12, MAFB, SLC2A6, xPOP7, C1orf144, SLC25A6, EIF1, ATP5B, ETFB, FBXW4, TTC9C, NAGA, MYO1G, GSTK1, UBE2S, AP2S1, DNPEP, TGFBI, LSM12, LIPA, RPML2, CITED2, FUCA1, NUP62, OXA1L, ASB13, NANS, CD68, TSSC4, COMMD4, F8A1, PCGF1, RAD23A, YY1, MRPL43, GPS1, ARL61P4, ALDOA, FAM54B, NMT1, ATP5J2, VPS11, CLPTM1, NMRAL1, FKSG30, RRAS, COMT, SNHG5, PSMB1, LRRC8D, HDLBP, SCAND1, CSF1R, C20orf4, TPI1, NAPSB, POLDIP2, SNX15, CTSH, TMED1, POLR3K, C22orf9, KLHDC3, LAIR1, PLXNB2, AGPAT3, VPS26B, COMMD9, AP1M1, GSS, EEF1D, MRPL23, ITGB2, C10orf56, ISCA1, PQBP1, SLC22A18, KRT10, C19orf54, COPZ1, WIPE, SIGLEC10, MGST3, UQCRC1, ASNA1, SCARB2, TSSC1, C1orf85, ADFP, BAK1, ENO1, POLR2E, DUSP3, IGSF2, H1FX, MRPS15, KCMF1, ZBED1, CRTAP, SUSD1, ALKBH7, GHITM, CENTA2, DYM, DPYSL2, NDFIP1, C2orf47, PLEKHF1, Cllorf75, ATOX1, PRICKLE4, PEPD, MDH2, SPNS1, JTV1, HNRNPUL1, ENO1L1, ILF3, RPUSD3, ACO2, CENPB, TCF7L2, MAPKAP1, G6PC3, DHPS, RTN1, RABGGTA, CAMTA1, KBRAS2, FARS1, ESD, PTDSS1, C3orf60, HLA-DQB1, NAGPA, XRCC1, NDUFS7, TTLL12, FH, FLAD1, RSU1, ALDH6A1, DCXR, RSL1D1, NUDC, DTD1, CNDP2, TRPV2, ZNF689, H6PD, RNF26, ABHD12, C17orf63, TP53, SUGT1, IDH3B, VDAC2, AlP, C8orf30A, CSNK2A1, HADH2, OCIAD1, TRIM44, PTBP1, SMARCB1, PSMD2, RNF5, EIF3M, NOB1, RALY, GATAD2A, COG2, SCAMP2, MTHFD1, AKR7A2, POLR3E, ECHS1, ACP5, RPL38, CCDC86, NUBP1, AHCYL2, TP53BP1, FTO, NOL5A, BSCL2, LRRC41, SEC31A, SCLY, MAP4, C4orfl4, FVT1, FAHD2A, HNRNPD, FAM50B, SRM, LARP1, ZNF777, FARSA, CALR, ZFP64, NUDT19, FGD2, GTPBP3, POLR1C, and PCMTD1.

According to the blood diagnosis method, because a biomarker is used as a diagnostic marker, it is possible to provide a diagnostic marker which is versatile and which can contribute to the improvements in dialysis treatment and the evaluation of clinical effects.

In the step for collecting blood samples, it is also possible to collect the blood samples using a dialysis machine.

A third aspect according to the present invention is a dialysis comprising a dialyzer to purify blood; and a blood sample collecting unit which collects a blood sample for making a diagnosis on blood collected from a patient using a biomarker, wherein the biomarker is identified in advance based on the correlation between the urea clear space (CS) or the cellular membrane clearance (Kc) and the profiles of mRNA or proteins.

According to the dialysis machine, because a biomarker is identified in advance based on the correlation between the urea clear space (CS) or the cellular membrane clearance (Kc) and the profiles of mRNA or proteins, it is possible to provide a diagnostic marker which is versatile and which can contribute to the improvements in dialysis treatment and the evaluation of clinical effects.

A fourth aspect according to the present invention is a dialysis machine comprising a dialyzer to purify blood; and a blood sample collecting unit which collects blood samples for making a diagnosis using a biomarker, wherein any one of the genes shown below or the combinations thereof is used as the biomarker which is identified from the profiles of mRNA or proteins:

TNFSF12, MAFB, SLC2A6, POP7, C1orf144, SLC25A6, EIF1, ATP5B, ETFB, FBXW4, TTC9C, NAGA, MYO1G, GSTK1, UBE2S, AP2S1, DNPEP, TGFBI, LSM12, LIPA, RPML2, CITED2, FUCA1, NUP62, OXA1L, ASB13, NANS, CD68, TSSC4, COMMD4, F8A1, PCGF1, RAD23A, YY1, MRPL43, GPS1, ARL61P4, ALDOA, FAM54B, NMT1, ATP5J2, VPS11, CLPTM1, NMRAL1, FKSG30, RRAS, COMT, SNHG5, PSMB1, LRRC8D, HDLBP, SCAND1, CSF1R, C20orf4, TPI1, NAPSB, POLDIP2, SNX15, CTSH, TMED1, POLR3K, C22orf9, KLHDC3, LAIR1, PLXNB2, AGPAT3, VPS26B, COMMD9, AP1M1, GSS, EEF1D, MRPL23, ITGB2, C10orf56, ISCA1, PQBP1, SLC22A18, KRT10, C19orf54, COPZ1, WIPI1, SIGLEC10, MGST3, UQCRC1, ASNA1, SCARB2, TSSC1, C1orf85, ADFP, BAK1, ENO1, POLR2E, DUSP3, IGSF2, H1FX, MRPS15, KCMF1, ZBED1, CRTAP, SUSD1, ALKBH7, GHITM, CENTA2, DYM, DPYSL2, NDFIP1, C2orf47, PLEKHF1, Cllorf75, ATOX1, PRICKLE4, PEPD, MDH2, SPNS1, JTV1, HNRNPUL1, ENO1L1, ILF3, RPUSD3, ACO2, CENPB, TCF7L2, MAPKAP1, G6PC3, DHPS, RTN1, RABGGTA, CAMTA1, KBRAS2, FARS1, ESD, PTDSS1, C3orf60, HLA-DQB1, NAGPA, XRCC1, NDUFS7, TTLL12, FH, FLAD1, RSU1, ALDH6A1, DCXR, RSL1D1, NUDC, DTD1, CNDP2, TRPV2, ZNF689, H6PD, RNF26, ABHD12, C17orf63, TP53, SUGT1, IDH3B, VDAC2, AlP, C8orf30A, CSNK2A1, HADH2, OCIAD1, TRIM44, PTBP1, SMARCB1, PSMD2, RNF5, EIF3M, NOB1, RALY, GATAD2A, COG2, SCAMP2, MTHFD1, AKR7A2, POLR3E, ECHS1, ACP5, RPL38, CCDC86, NUBP1, AHCYL2, TP53BP1, FTO, NOL5A, BSCL2, LRRC41, SEC31A, SCLY, MAP4, C4orfl4, FVT1, FAHD2A, HNRNPD, FAM50B, SRM, LARP1, ZNF777, FARSA, CALR, ZFP64, NUDT19, FGD2, GTPBP3, POLR1C, and PCMTD1.

According to the dialysis machine, because a biomarker is used as a diagnostic marker, it is possible to provide a diagnostic marker which is versatile and which can contribute to the improvements in dialysis treatment and the evaluation of clinical effects.

According to the blood diagnosis method of the present invention, because a biomarker is identified in advance based on the correlation between the urea clear space (CS) or the cellular membrane clearance (Kc) and the profiles of mRNA or proteins, it is possible to provide a diagnostic marker which is versatile and which can contribute to the improvements in dialysis treatment and the evaluation of clinical effects.

According to the blood diagnosis method of the present invention, because a biomarker is used as a diagnostic marker, it is possible to provide a diagnostic marker which is versatile and which can contribute to the improvements in dialysis treatment and the evaluation of clinical effects.

According to the dialysis machine of the present invention, because a biomarker is identified in advance based on the correlation between the urea clear space (CS) or the cellular membrane clearance (Kc) and the profiles of mRNA or proteins, it is possible to provide a diagnostic marker which is versatile and which can contribute to the improvements in dialysis treatment and the evaluation of clinical effects.

According to the dialysis machine of the present invention, because a biomarker is used as a diagnostic marker, it is possible to provide a diagnostic marker which is versatile and which can contribute to the improvements in dialysis treatment and the evaluation of clinical effects.

### BRIEF DESCRIPTION OF THE DRAWINGS

FIG. 1 is a diagram showing an identified group of genes.

FIG. 2 is a diagram showing an identified group of genes.

FIG. 3 is a diagram showing an identified group of genes.

FIG. 4 is a diagram showing an identified group of genes.

FIG. 5 is a diagram illustrating a configuration example of a dialysis machine.

### DESCRIPTION OF THE REFERENCE SYMBOLS

1: Dialysis machine;
11: Dialyzer;
13: Valve (blood sample collecting means).

### DETAILED DESCRIPTION OF THE INVENTION

Hereinafter, one embodiment of a blood diagnosis method according to the present invention will be described.

The present invention is based on the finding that profiles of a specific group of mRNA or proteins in blood samples before and after dialysis of a dialysis patient have a correlation with conventional diagnostic markers. The present inventor discovered that it is possible to obtain useful correlation data between clinical conditions and gene diagnosis information by narrowing the range of the genes whose expression levels in the form of mRNA or proteins exhibit particularly strong correlation. By using the group of mRNA or proteins having a high correlation with the patient's primary disease or a specific clinical parameter as a diagnostic marker, it is possible to provide a diagnostic tool which is versatile and is simple and easy to use.

A blood diagnosis method according to the present embodiment is performed in the following procedure.

(1) A blood sample before dialysis is collected from a dialysis patient.
(2) Dialysis is performed using a dialysis machine.
(3) A blood sample after dialysis is collected from the dialysis patient.
(4) Profiles of mRNA or proteins are obtained from the blood samples collected before and after the dialysis.
(5) Diagnosis is made based on the profiles of mRNA or proteins by using a specific group of mRNA or proteins as a marker.

As described above, in the blood diagnosis method according to the present embodiment, a specific group of mRNA or proteins in the blood is used as a marker. For the group of genes whose expression products (i.e., mRNA or proteins) are used as markers, those having a high correlation with the urea clear space (CS) or the cellular membrane clearance for urea (Kc) are selected.

These groups of genes can be selected from a variety of viewpoints. For example, a group of genes which are up-regulated and a group of genes which are down-regulated by the dialysis in the blood samples of a chronic hepatitis patient group can be identified as “chronic hepatitis biomarkers”. A cartridge can be constituted by arranging probes for these groups of genes. In this case, probes for a group of genes which do not show changes in the level of expression (i.e., not particularly up-regulated or down-regulated) due to the dialysis among all patients can be used as control probes.

Similarly, by identifying an appropriate biomarker specific to a group of diabetic nephropathy patients or for the entire group of patients with renal disease, the biomarkers can be selected for use as a“diabetic nephropathy biomarker” or“biomarker for all renal diseases” in the blood diagnosis method or the dialysis machine according to the present invention. By using these markers, it is possible to estimate the severity of the medical condition or to identify the primary disease.

Moreover, by appropriately selecting a group of genes whose expression levels have a high correlation with existing clinical parameters and identifying them as biomarkers, it is possible to make a diagnosis using the biomarkers instead of using the clinical parameters. For example, a group of genes whose expression levels are correlated with the creatinine generation rate, which is a useful clinical parameter as a determination index of a treatment effect or a patient's nutritive condition, can be used as a biomarker. Similarly, a group of genes whose expression levels are correlated with another indicator which has been used as a conventional marker can be used as a biomarker.

By using a group of genes correlated with an index indicating a nutritive condition such as PEM (Protein Energy Malnutrition) as a biomarker, it is possible to obtain a new set of indicators providing a different perspective from that of the conventional diagnostic markers. In addition, by using a group of genes correlated with an inflammatory cytokine as a biomarker, it is possible to understand the pathological condition of uremia of a patient with end-stage renal failure. Moreover, through an appropriate selection of biomarkers for infectious diseases such as pneumonia or bronchitis specific to the elderly, it is possible to understand, for example, the liability thereof.

Furthermore, by appropriately selecting a group of genes, as a biomarker, whose expression levels have a high correlation with the type of dialysis membrane to be used, it is possible to use the biomarker as an indicator for appropriately selecting the dialysis membrane.

The identification of biomarkers and the diagnosis using the biomarkers can be carried out based on a general statistical technique. Through a feedback process regarding the patient's clinical data obtained by performing a diagnosis using a biomarker, it is possible to continuously improve diagnostic accuracy. It is also possible to add a biomarker corresponding to a new clinical indicator or to add a new biomarker corresponding to the same clinical indicator.

Renal anemia can greatly affect the quality of life (QOL) of dialysis patients. By optimizing the indicators for dialysis dose such as Kc/CS, it is possible to up-regulate the expression of genes (in the form of mRNA) that are related to anemia. By using biomarkers for the renal anemia, it is possible to reduce erithropoietin dose, which is also useful in terms of medical economy.

The blood diagnosis method according to the present invention using a biomarker is performed by conducting a gene analysis using a gene diagnosis system directly on a blood sample or on the blood sample having been subjected to a pretreatment.

As described above, in the blood diagnosis method according to the present invention, the following effects can be achieved by appropriately selecting the biomarkers in advance which are correlated with the clinical data.

(1) It is possible to appropriately select a dialysis membrane in accordance with the patient's condition. Since the diagnosis can be made rapidly by using a biomarker, it is possible to adequately select an appropriate dialysis membrane on every occasion.
(2) It is easy to find out the primary disease of a chronic dialysis patient.
(3) It is possible to obtain diagnostic evaluation and dialysis treatment reflecting individual differences.
(4) It is possible to prevent complications such as infectious diseases. By using a biomarker serving as an indicator of the complications, it is possible to obtain an assessment regarding the complications and to achieve an adequate dialysis treatment, which has been difficult to derive from the conventional diagnostic markers.
(5) It is possible to establish an adequate treatment plan for the patients by identifying the cause of the disease. For example, by using a biomarker corresponding to clinical data, it is possible to determine the causes of chronic nephritis, renal disease derived from diabetes, and the like.
(6) It is possible to monitor a patient's nutritive condition. As described above, by using a group of genes correlated with an index indicating a nutritive condition such as PEM (Protein Energy Malnutrition) as a biomarker, it is possible to improve the medical conditions of patients through the improvements of their nutritive conditions.
(7) Since the relationship between patients' primary disease or their medical conditions and a treatment effect due to the dialysis on the patients can be elucidated by accumulating the data on correlations between the clinical data and the profiles of mRNA or proteins, it is possible to adequately determine when to start the treatment by dialysis. Accordingly, it may be possible to prolong the period of predialysis treatment.

As described above, in the blood diagnosis method according to the present invention, a group of genes whose expression levels are highly correlated with the urea clear space (CS) or the cellular membrane clearance for urea (Kc) is used as a marker. In particular, the relationship between the urea clear space (CS) and the prolonging of life has been accepted by Japanese Society of Nephrology. Therefore, the identification of a group of genes whose expression is highly correlated with the urea clear space (CS) is equivalent to the identification of a group of genes related with the prolonging of life, through an intermediate marker in the form of the urea clear space (CS).

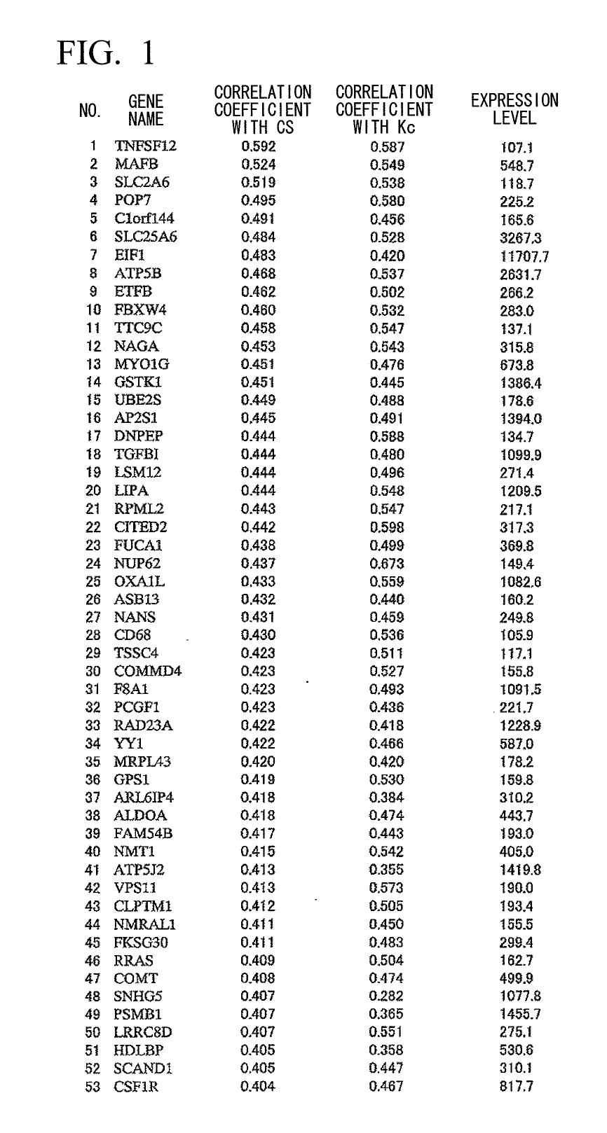

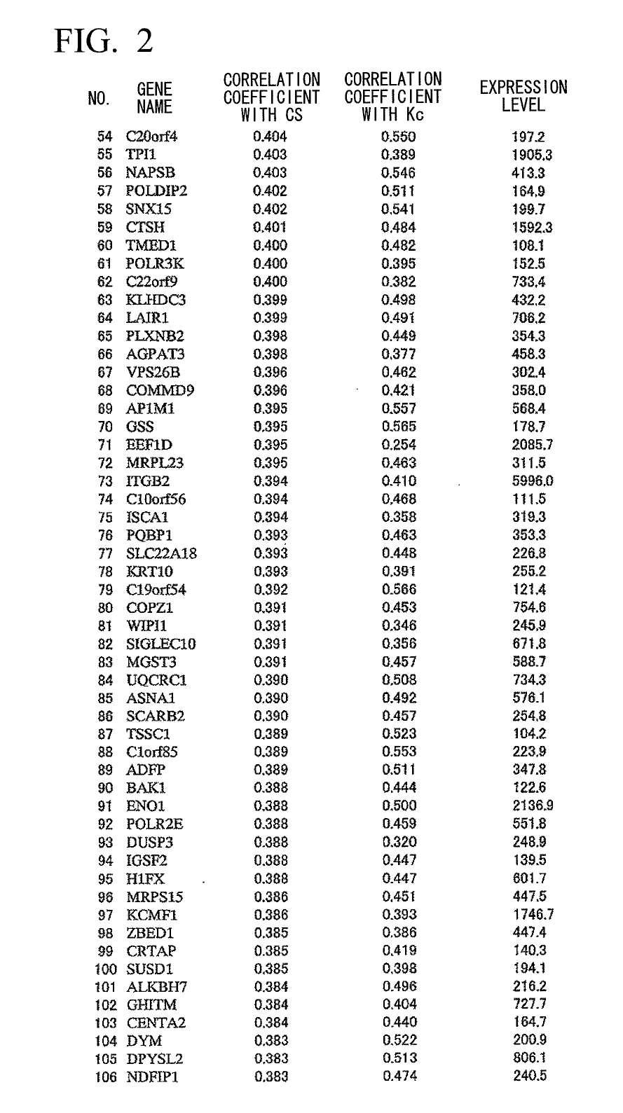

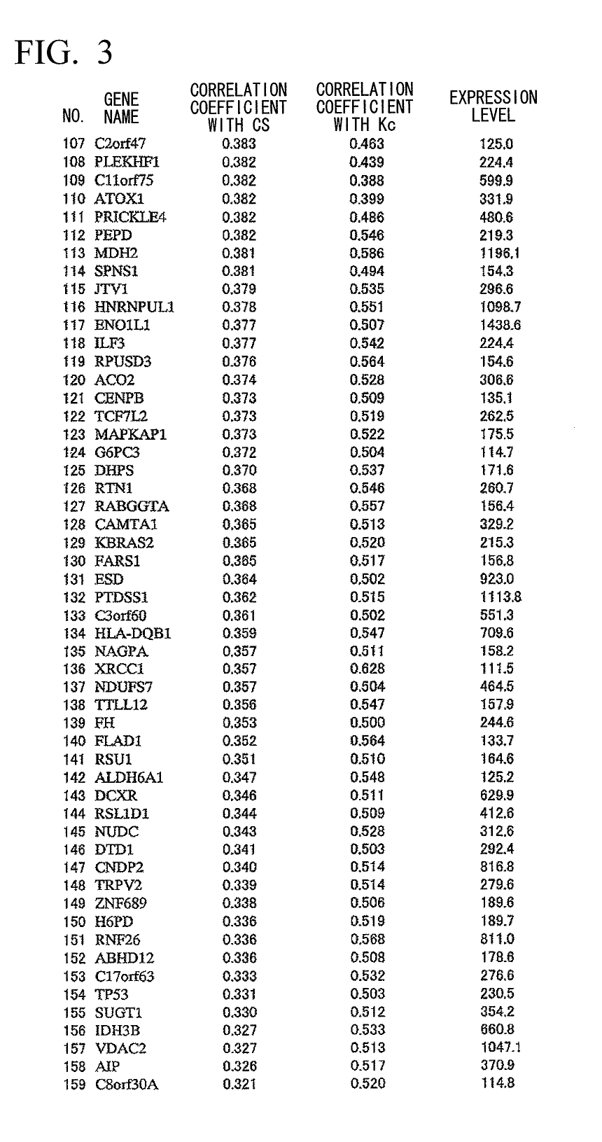

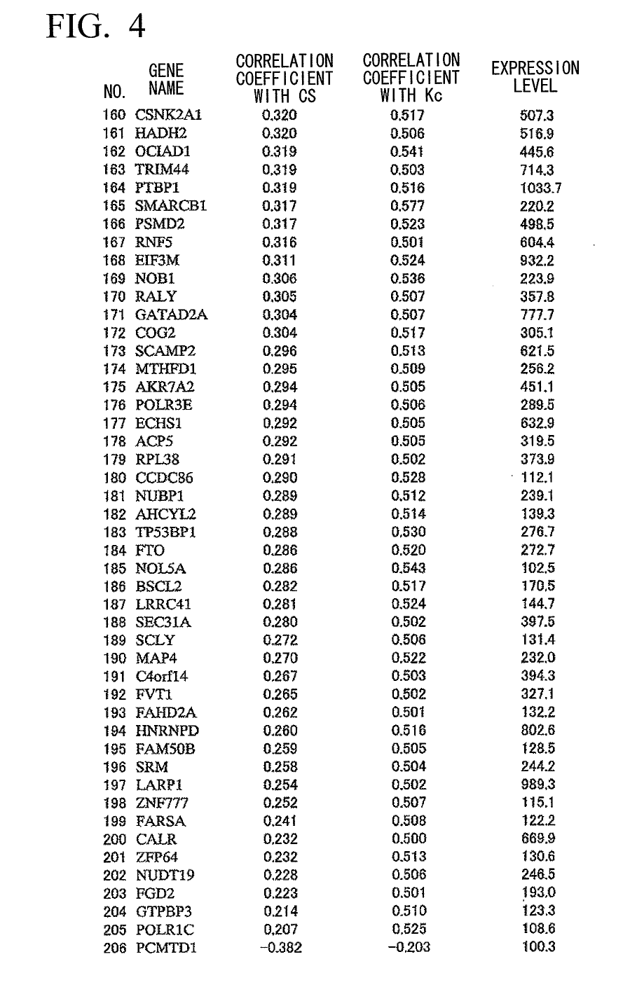

FIGS. 1 to 4 show a group of genes identified by the inventors of the present invention as having a high correlation with the urea clear space (CS) or the cellular membrane clearance for urea (Kc). In FIGS. 1 to 4, genes are listed in descending order of correlation coefficient with the urea clear space (CS), and the correlation coefficient with the cellular membrane clearance for urea (Kc) as well as the level of expression for each gene is shown.

A group of genes whose expression is highly correlated with the urea clear space (CS) or the cellular membrane clearance (Kc) and which is expressed to a certain level can be used as a marker. Examples of the genes selected from such viewpoints include TNFSF12, MAFB, SLC2A6, POP7, C1orfl44, SLC25A6, EIF1, ATP5B, ETFB, FBXW4, TTC9C, NAGA, MYO1G, GSTK1, UBE2S, AP2S1, DNPEP, TGFBI, LSM12, L1PA, RPML2, CITED2, FUCA1, NUP62, OXA1L, ASB13, NANS, CD68, TSSC4, COMMD4, F8A1, PCGF1, RAD23A, YY1, MRPL43, GPS1, ARL61P4, ALDOA, FAM54B, NMT1, ATP5J2, VPS11, CLPTM1, NMRAL1, FKSG30, RRAS, COMT, SNHG5, PSMB1, LRRC8D, HDLBP, SCAND1, CSF1R, C20orf4, TPI1, NAPSB, POLDIP2, SNX15, CTSH, TMED1, POLR3K, C22orf9, KLHDC3, LAIR1, PLXNB2, AGPAT3, VPS26B, COMMD9, AP1M1, GSS, EEF1D, MRPL23, ITGB2, C10orf56, ISCA1, PQBP1, SLC22A18, KRT10, C19orf54, COPZ1, WIPI1, SIGLEC10, MGST3, UQCRC1, ASNA1, SCARB2, TSSC1, C1orf85, ADFP, BAK1, ENO1, POLR2E, DUSP3, IGSF2, H1FX, MRPS15, KCMF1, ZBED1, CRTAP, SUSD1, ALKBH7, GHITM, CENTA2, DYM, DPYSL2, NDFIP1, C2orf47, PLEKHF1, Cllorf75, ATOX1, PRICKLE4, PEPD, MDH2, SPNS1, JTV1, HNRNPUL1, ENO1L1, ILF3, RPUSD3, ACO2, CENPB, TCF7L2, MAPKAP1, G6PC3, DHPS, RTN1, RABGGTA, CAMTA1, KBRAS2, FARS1, ESD, PTDSS1, C3orf60, HLA-DQB1, NAGPA, XRCC1, NDUFS7, TTLL12, FH, FLAD1, RSU1, ALDH6A1, DCXR, RSL1D1, NUDC, DTD1, CNDP2, TRPV2, ZNF689, H6PD, RNF26, ABHD12, C17orf63, TP53, SUGT1, IDH3B, VDAC2, AlP, C8orf30A, CSNK2A1, HADH2, OCIAD1, TRIM44, PTBP1, SMARCB1, PSMD2, RNF5, EIF3M, NOB1, RALY, GATAD2A, COG2, SCAMP2, MTHFD1, AKR7A2, POLR3E, ECHS1, ACP5, RPL38, CCDC86, NUBP1, AHCYL2, TP53BP1, FTO, NOL5A, BSCL2, LRRC41, SEC31A, SCLY, MAP4, C4 orf14, FVT1, FAHD2A, HNRNPD, FAM50B, SRM, LARP1, ZNF777, FARSA, CALR, ZFP64, NUDT19, FGD2, GTPBP3, POLR1C, and PCMTD1.

According to the blood diagnosis method of the present invention, since the blood itself which is dialyzed is used as a sample, it is possible to efficiently determine the effects of dialysis in comparison with the diagnosis made based on other clinical data. In addition, since the effect of dialysis is rapidly reflected in the blood sample, it is possible to make a rapid diagnosis.

Moreover, since the expression of mRNA or proteins is promoted by the stimuli when the blood passes through a dialysis membrane, it is possible to perform the gene analysis more effectively.

Further, according to the blood diagnosis method of the present invention, it is possible to determine a patient's inflammatory condition, nutritive condition, and sarcolysis condition by appropriately selecting a biomarker. Furthermore, it is possible to determine the state of cytokine production and thus consequently to determine refractoriness to erythropoietin, resistance to insulin, and rapid enhancement of adipocytokine secretion.

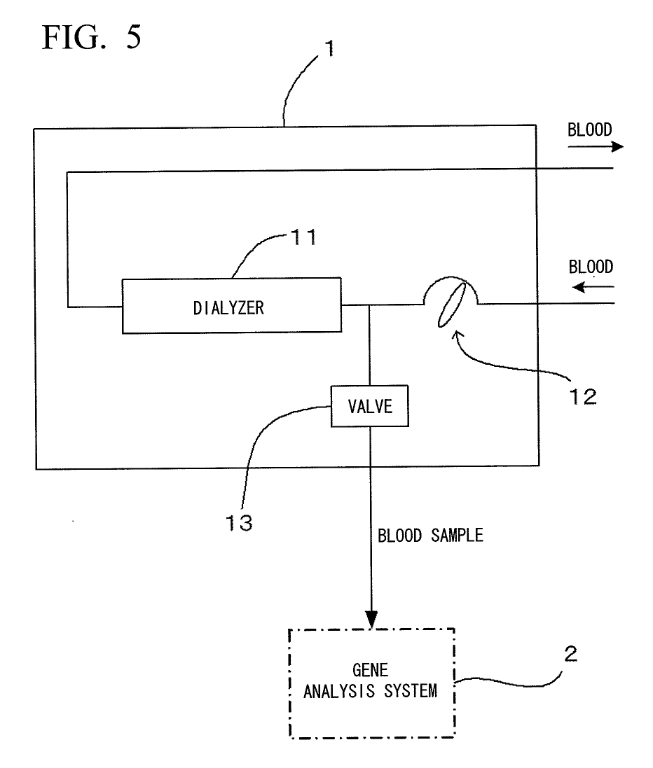

FIG. 5 is a diagram illustrating a configuration example of a dialysis machine.

A blood sample can be collected directly from the dialysis machine 1. Patient's blood passes through a liquid transferring device 12 and a dialyzer 11 of the dialysis machine 1 in this order, and is then returned to the patient's body. As shown in FIG. 5, a valve 13 for collecting a blood sample is disposed in front of the dialyzer 11, and it is thus possible to collect a patient's blood for diagnosis and make a diagnosis as described above by using a biomarker by opening the valve 13 at the time of starting or ending the dialysis. It should be noted that in the present invention, the expression “before and after dialysis” means that a blood sample is collected not only before the start of the dialysis operation and after the completion of the dialysis operation, but a blood sample is also collected several times during the dialysis operation. Accordingly, a blood sample may be collected during the dialysis. By collecting a blood sample during dialysis and then examining the collected blood sample by the use of a gene analysis system 2, it is possible to monitor the patient's condition over time during the dialysis.

By using the dialysis machine 1 shown in FIG. 5, it is possible to collect a blood sample without any burden on the patients. In addition, no labor is required for collecting a blood sample. It is also possible to make the blood sample collected by the dialysis machine 1 automatically introduced to the gene analysis system 2. In this case, it is possible to suppress the amount of blood samples required for the analysis.

As described above, according to the blood diagnosis method and the dialysis machine of the present invention, since a diagnosis of a collected blood sample is made on the basis of a biomarker, it is possible to obtain a diagnosis result that is versatile and useful through a simple and easy procedure.

The present invention can be widely used in blood diagnosis method or the like in which a diagnosis is made based on blood collected from a dialysis patient.

While preferred embodiments of the present invention have been described and illustrated above, it should be understood that these are exemplary of the invention and are not to be considered as limiting.

1. A blood diagnosis method in which a diagnosis is made using blood collected from a dialysis patient, the method comprising:
a step for identifying a biomarker in advance based on a correlation between a urea clear space (CS) or a cellular membrane clearance (Kc), and gene expression profiles;
a step for collecting a blood sample from a dialysis patient before and after dialysis; and
a step for making a diagnosis regarding the collected blood sample based on the identified biomarker.

2. A blood diagnosis method in which a diagnosis is made using blood collected from a dialysis patient, the method comprising:
a step for collecting a blood sample from a dialysis patient before and after dialysis; and
a step for making a diagnosis regarding the collected blood sample based on a biomarker,
wherein any one of the genes shown below or the combinations thereof is used as the biomarker which is identified from the profiles of mRNA or proteins:
TNFSF12, MAFB, SLC2A6, POP7, C1orf144, SLC25A6, EIF1, ATP5B, ETFB, FBXW4, TTC9C, NAGA, MYO1G, GSTK1, UBE2S, AP2S1, DNPEP, TGFBI, LSM12, L1PA, RPML2, CITED2, FUCA1, NUP62, OXA1L, ASB13, NANS, CD68, TSSC4, COMMD4, F8A1, PCGF1, RAD23A, YY1, MRPL43, GPS1, ARL61P4, ALDOA, FAM54B, NMT1, ATP5J2, VPS11, CLPTM1, NMRAL1, FKSG30, RRAS, COMT, SNHG5, PSMB1, LRRC8D, HDLBP, SCAND1, CSF1R, C20orf4, TPI1, NAPSB, POLDIP2, SNX15, CTSH, TMED1, POLR3K, C22orf9, KLHDC3, LAIR1, PLXNB2, AGPAT3, VPS26B, COMMD9, AP1M1, GSS, EEF1D, MRPL23, ITGB2, C10orf56, ISCA1, PQBP1, SLC22A18, KRT10, C19orf54, COPZ1, WIPI1, SIGLEC10, MGST3, UQCRC1, ASNA1, SCARB2, TSSC1, C1orf85, ADFP, BAK1, ENOL, POLR2E, DUSP3, IGSF2, H1FX, MRPS15, KCMF1, ZBED1, CRTAP, SUSD1, ALKBH7, GHITM, CENTA2, DYM, DPYSL2, NDFIP1, C2orf47, PLEKHF1, C11orf75, ATOX1, PRICKLE4, PEPD, MDH2, SPNS1, JTV1, HNRNPUL1, ENO1L1, ILF3, RPUSD3, ACO2, CENPB, TCF7L2, MAPKAP1, G6PC3, DHPS, RTN1, RABGGTA, CAMTA1, KBRAS2, FARS1, ESD, PTDSS1, C3orf60, HLA-DQB1, NAGPA, XRCC1, NDUFS7, TTLL12, FH, FLAD1, RSU1, ALDH6A1, DCXR, RSL1D1, NUDC, DTD1, CNDP2, TRPV2, ZNF689, H6PD, RNF26, ABHD12, C17orf63, TP53, SUGT1, IDH3B, VDAC2, AIP, C8orf30A, CSNK2A1, HADH2, OCIAD1, TRIM44, PTBP1, SMARCB1, PSMD2, RNF5, EIF3M, NOB1, RALY, GATAD2A, COG2, SCAMP2, MTHFD1, AKR7A2, POLR3E, ECHS1, ACP5, RPL38, CCDC86, NUBP1, AHCYL2, TP53BP1, FTO, NOL5A, BSCL2, LRRC41, SEC31A, SCLY, MAP4, C4orf14, FVT1, FAHD2A, HNRNPD, FAM50B, SRM, LARP1, ZNF777, FARSA, CALR, ZFP64, NUDT19, FGD2, GTPBP3, POLR1C, and PCMTD1.

3. The blood diagnosis method according to claim 1,
wherein in the step for collecting a blood sample, the blood sample is collected using a dialysis machine.

4. A dialysis machine comprising:
a dialyzer to purify blood; and
a blood sample collecting unit which collects a blood sample for making a diagnosis on blood collected from a patient using a biomarker,
wherein the biomarker is identified in advance based on a correlation between a urea clear space (CS) or a cellular membrane clearance (Kc), and profiles of mRNA or proteins.

5. A dialysis machine comprising:
a dialyzer to purify blood; and
a blood sample collecting unit which collects a blood sample for making a diagnosis on blood collected from a patient using a biomarker,
wherein any one of the genes shown below or the combinations thereof is used as the biomarker which is identified from the profiles of mRNA or proteins:
TNFSF12, MAFB, SLC2A6, POP7, C1orf144, SLC25A6, EIF1, ATP5B, ETFB, FBXW4, TTC9C, NAGA, MYO1G, GSTK1, UBE2S, AP2S1, DNPEP, TGFBI, LSM12, L1PA, RPML2, CITED2, FUCA1, NUP62, OXA1L, ASB13, NANS, CD68, TSSC4, COMMD4, F8A1, PCGF1, RAD23A, YY1, MRPL43, GPS1, ARL61P4, ALDOA, FAM54B, NMT1, ATP5J2, VPS11, CLPTM1, NMRAL1, FKSG30, RRAS, COMT, SNHG5, PSMB1, LRRC8D, HDLBP, SCAND1, CSF1R, C20orf4, TPI1, NAPSB, POLDIP2, SNX15, CTSH, TMED1, POLR3K, C22orf9, KLHDC3, LAIR1, PLXNB2, AGPAT3, VPS26B, COMMD9, AP1M1, GSS, EEF1D, MRPL23, ITGB2, C10orf56, ISCA1, PQBP1, SLC22A18, KRT10, C19orf54, COPZ1, WIPI1, SIGLEC10, MGST3, UQCRC1, ASNA1, SCARB2, TSSC1, C1orf85, ADFP, BAK1, ENOL, POLR2E, DUSP3, IGSF2, H1FX, MRPS15, KCMF1, ZBED1, CRTAP, SUSD1, ALKBH7, GHITM, CENTA2, DYM, DPYSL2, NDFIP1, C2orf47, PLEKHF1, C11orf75, ATOX1, PRICKLE4, PEPD, MDH2, SPNS1, JTV1, HNRNPUL1, ENO1L1, ILF3, RPUSD3, ACO2, CENPB, TCF7L2, MAPKAP1, G6PC3, DHPS, RTN1, RABGGTA, CAMTA1, KBRAS2, FARS1, ESD, PTDSS1, C3orf60, HLA-DQB1, NAGPA, XRCC1, NDUFS7, TTLL12, FH, FLAD1, RSU1, ALDH6A1, DCXR, RSL1D1, NUDC, DTD1, CNDP2, TRPV2, ZNF689, H6PD, RNF26, ABHD12, C17orf63, TP53, SUGT1, IDH3B, VDAC2, AIP, C8orf30A, CSNK2A1, HADH2, OCIAD1, TRIM44, PTBP1, SMARCB1, PSMD2, RNF5, EIF3M, NOB1, RALY, GATAD2A, COG2, SCAMP2, MTHFD1, AKR7A2, POLR3E, ECHS1, ACP5, RPL38, CCDC86, NUBP1, AHCYL2, TP53BP1, FTO, NOL5A, BSCL2, LRRC41, SEC31A, SCLY, MAP4, C4orf14, FVT1, FAHD2A, HNRNPD, FAM50B, SRM, LARP1, ZNF777, FARSA, CALR, ZFP64, NUDT19, FGD2, GTPBP3, POLR1C, and PCMTD1.

6. The dialysis machine according to claim 4,
wherein the blood sample collecting unit is disposed in front of the dialyzer and the blood sample is collected before being transferred to the dialyzer.

7. The blood diagnosis method according to claim 2,
wherein in the step for collecting a blood sample, the blood sample is collected using a dialysis machine.

8. The dialysis machine according to claim 5,
wherein the blood sample collecting unit is disposed in front of the dialyzer and the blood sample is collected before being transferred to the dialyzer.
